(19) United States
(12) Patent Application Publication
(10) Pub. No.: US 2013/0029355 A1
(43) Pub. Date: Jan. 31, 2013

(54) MULTIPLE ANALYSIS DEVICE AND METHOD FOR ANALYZING CANCER CELLS IN BLOOD

(71) Applicants: Seong-Mok CHO, Daejeon (KR); Moon Youn Jung, Daejeon (KR); Dae-Sik Lee, Daejeon (KR); Jeong Won Park, Daejeon (KR)

(72) Inventors: Seong-Mok CHO, Daejeon (KR); Moon Youn Jung, Daejeon (KR); Dae-Sik Lee, Daejeon (KR); Jeong Won Park, Daejeon (KR)

(73) Assignee: Electronics and Telecommunications Research Institute, Daejeon (KR)

(21) Appl. No.: 13/533,918

(22) Filed: Jun. 26, 2012

(30) Foreign Application Priority Data
Jul. 28, 2011 (KR) 10-2011-0075412
Feb. 17, 2012 (KR) 10-2012-0016463

(51) Int. Cl. G01N 27/72; C12M 1/42; B82Y 25/00; B82Y 5/00

(52) U.S. Cl. 435/7.23; 435/287.2; 977/773; 977/838; 977/920; 977/904

(57) ABSTRACT

Provided are a multiple analysis device and a method of analyzing cancer cells in blood using the device. In this device and method, it can analyze the cancer cells along cancer kinds by using the magnetic nanoparticles combined to the markers of the cancer cells and the difference of the magnetic fields of them.

### CROSS-REFERENCE TO RELATED APPLICATIONS

This U.S. non-provisional patent application claims priority under 35 U.S.C. §119 of Korean Patent Application No. 10-2011-0075412, filed on Jul. 28, 2011, and Korean Patent Application No. 10-2012-0016463, filed on Feb. 17, 2012, the entire contents of which are hereby incorporated by reference.

### BACKGROUND

The present disclosure herein relates to a multiple analysis device and a method for analyzing cancer cells in blood.

It is required to separate components in cells or cell types as tools for a final objection or other analyses in diagnosis, remedy and study fields of a medicinal discipline. For example, it is needed to analyze a cancer cell. Cancer cells in blood are commonly named as cancer cells existing in peripheral blood of a cancer patient and fall off from an original carcinomatous focus or transition focus. These cancer cells in blood are expected as influential biomarkers for cancer diagnosis, remedy convalescence analysis, fine transition analysis and etc. Furthermore, the analysis of a cancer cell in blood has an advantage that this analysis is a non-invasive method in comparison with a conventional cancer diagnosis method. Therefore, this analysis is very brightly prospected as a future cancer diagnosis method. However, the distribution ratio of the cancer cell in blood is very low. For example, the distribution ratio of the cancer cell in blood is about one cancer cell per the total billion cells or about one cancer cell per about 10⁶˜10⁷ leukocytes. Therefore, the precise analysis is very difficult and an ingenious analysis method is required.

There have been studied many methods for separating cancel cells in blood. However, in conventional methods, the test time takes long and the methods only show the existence, the nonexistence and/or the number of the cancer cells. It is difficult to analyze the kind of the cancer. Furthermore, there is an interference problem by non-specifically combined blood corpuscle.

### SUMMARY

The present disclosure provides a multiple analysis device of various materials as well as biomaterials.

The present disclosure provides a method for analyzing cancer cells in blood.

Embodiments of the inventive concept provide a multiple analysis device including: a magnetophoresis separation part including a solution inlet where a solution is injected, a saline solution inlet where a saline solution is injected, a channel connected to both the solution inlet and the saline solution inlet, at least one ferromagnetic pattern disposed below a bottom of the channel and extending in a direction crossing a flow of the solution, a first outlet passageway where a first kind material particle contained in the solution is output, connected to the channel, and a second outlet passageway where a second kind material particle is output, connected to the channel; a magnetic field measuring part which measures a magnetic field of the second kind material particle and is connected to the second outlet passageway; and an analysis/discrimination part which analyzes a magnetic field of the second kind material and discriminates the second kind material particle.

The second kind material particle may have a second magnetization magnitude and the first kind material particle may have a first magnetization magnitude smaller than the second magnetization magnitude.

The solution may contain a plurality of the second kind material particles whose magnetization magnitudes are bigger than the first magnetization magnitude and different from each other, and the magnetic field measuring part continuously may measure magnetic fields of the second kind material particle.

The solution may be blood. The first kind material particle may be a normal cell. The second kind material particles may be cancer cells whose kinds are different from each other.

Each of the cancer cells may include a different number of markers.

The device may further include at least one first permanent magnet adjacent to the channel.

The magnetic field measuring part may include a giant magnetoresistance sensor.

The device may further include at least one second permanent magnet disposed in front of the magnetic field measuring part and adjacent to the second outlet passageway.

Embodiments of the inventive concept provide a method of analyzing a cancer cell in blood, including: mixing a blood sample for test and a magnetic nanoparticle combined with an antibody which can specifically be reacted to a cancer cell, thereby forming a mixed solution containing a cancer cell combined with the magnetic nanoparticle; separating the mixed solution into normal cells and cancer cells using a magnetophoresis method; continuously measuring magnetic fields of the cancer cells; and analyzing/discriminating the cancer cells into cancer kinds by using the magnetic fields of the cancer cells.

The method may further include aligning directions of the magnetic fields of the magnetic nanoparticles combined to the cancer cells by using a permanent magnet, before continuously measuring the magnetic fields of the cancer cells.

The method may further include removing a magnetic nanoparticle which is not combined to the cancer cell.

The removing of a magnetic nanoparticle which is not combined to the cancer cell may include: obtaining a precipitate by centrifuging the mixed solution; and redispersing the precipitate by mixing the precipitate with a saline solution.

### BRIEF DESCRIPTION OF THE DRAWINGS

The accompanying drawings are included to provide a further understanding of the inventive concept, and are incorporated in and constitute a part of this specification. The drawings illustrate exemplary embodiments of the inventive concept and, together with the description, serve to explain principles of the inventive concept. In the drawings:

FIG. 1 is a flowchart illustrating a method of analyzing cancer cells in blood according to an example of the inventive concept;

FIG. 2 shows materials particles contained in a mixed solution according to an example of the inventive concept;

FIG. 3 is a schematic plan view showing a multiple analysis device according to an example of the inventive concept;

FIG. 4A is a detailed plan view of a magnetophoresis separation part contained in the multiple separation device of FIG. 2;

FIG. 4B is a cross-sectional view taken along the line A-A′ of FIG. 4A.

FIG. 4C shows movement of a material particle at the magnetophoresis separation part; and

FIG. 5 is a schematic plan view showing a multiple separation device according to an example of the inventive concept.

### DETAILED DESCRIPTION OF THE EMBODIMENTS

Preferred embodiments of the present invention will be described below in more detail. The invention may, however, be embodied in many different forms and should not be construed as being limited to the embodiments set forth herein; rather, these embodiments are provided so that this disclosure will be thorough and complete, and will fully convey the concept of the invention to those skilled in the art.

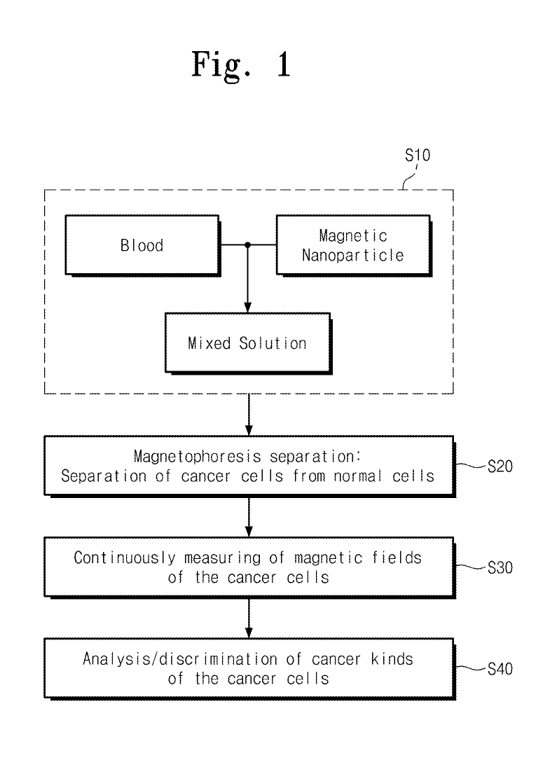

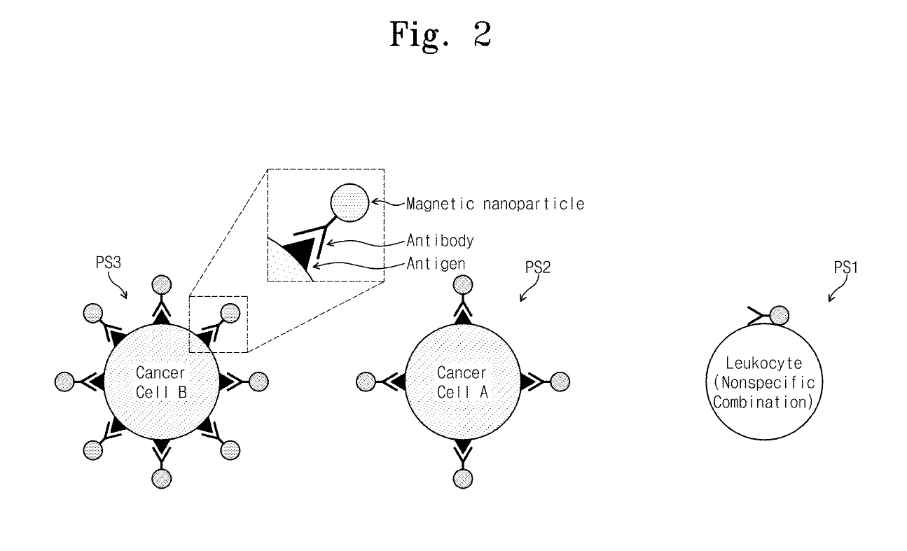

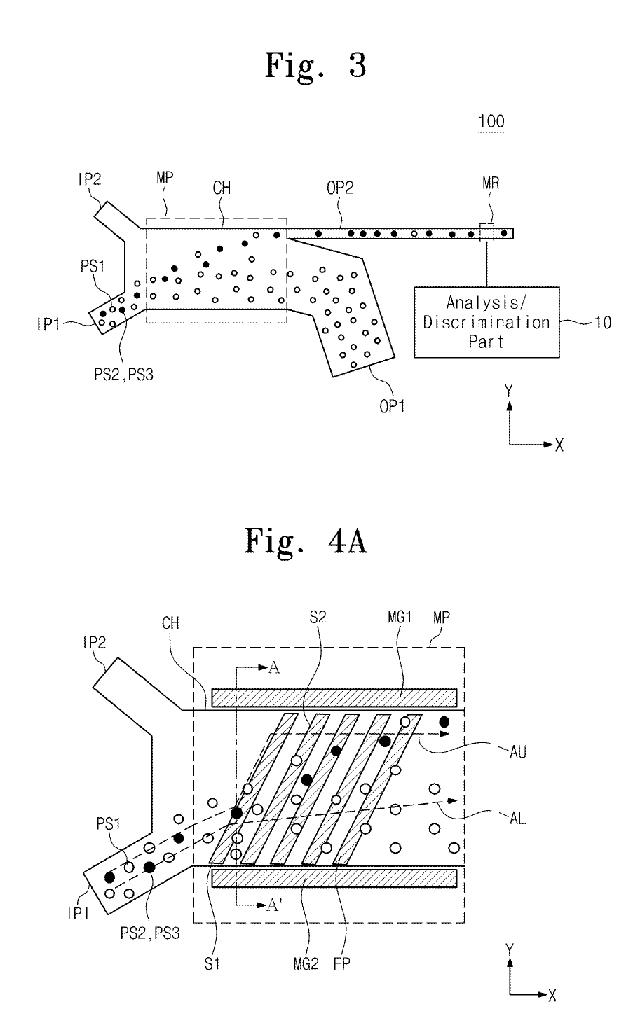

FIG. 1 is a flowchart illustrating a method of analyzing cancer cells in blood according to an example of the inventive concept. FIG. 2 shows materials particles contained in a mixed solution according to an example of the inventive concept. FIG. 3 is a schematic plan view showing a multiple analysis device according to an example of the inventive concept.

Referring to FIGS. 1 and 2, in a method of separating cancer cells in blood according to an example of the inventive concept, a blood for a test is mixed with magnetic nanoparticles combined with an antibody which can specifically react to a cancer cell, thereby forming a mixed solution (A first step, S10) containing cancer cells combined with the magnetic nanoparticle. The blood may contain a normal cell (a first kind material particle, PS1) such as a leukocyte, a cancer cell A (a second kind material particle, PS2) and a cancer cell B (a third material particle, PS3) which are different each other. If the kinds of the cancer cells PS2 and PS3 are different, the numbers of the markers (For example, antigen) expressed to the cancer cells are also different. In a case of EpCAM (epithelial cellular adhesion molecule) marker, the number of EpCAM expression per a cell of a breast cancer cell SKBr-3 is about 500,000, the number of EpCAM expression per a cell of a breast cancer cell PC3-9 is about 50,000, and the number of EpCAM expression per a cell of a bladder cancer cell T-24 is about 2,000. Like this, there is a big difference of the number of the marker expressed per one cancer cell. Therefore, if an antibody which can specifically reacted to the EpCAM is combined with magnetic nanoparticles and these magnetic nanoparticles and blood of a cancer patient are mixed, there is a big difference of a number of the magnetic nanoparticles combined to the cancer cell according to a cancer kind.

This difference of the number of the magnetic nanoparticles combined per a cell can be used for separating cancer cells and discriminating cancer kinds using magnetic field. As the number of the magnetic nanoparticles is increased, a magnetization magnitude is increased. The magnetic nanoparticle can be non-specifically combined to a normal cell such as leukocyte. However, the number of the magnetic nanoparticle combined to the leukocyte can be remarkably small than that of the magnetic nanoparticle combined to markers of cancer cells. The number of the magnetic nanoparticles combined to the second kind material particle PS2 is more than that of the magnetic nanoparticles combined to the first kind material particle PS1 but less than that of the magnetic nanoparticles combined to the third kind material particle PS3. If the first kind material particle PS1, the second kind material particle PS2 and the third kind material particle PS3 have a first magnetization magnitude, a second magnetization magnitude and a third magnetization magnitude, respectively, the second magnetization magnitude is bigger than the first magnetization magnitude and smaller than the third magnetization magnitude. The mixed solution composed of the blood containing the magnetic nanoparticles can be analyzed and discriminated by using a multiple analysis device 100 of FIG. 3. Before injecting the mixed solution into the multiple analysis device 100, a magnetic nanoparticle which is not combined to the cancer cell can be removed. At this time, the removing of a magnetic nanoparticle which is not combined to the cancer cell may include: obtaining a precipitate by centrifuging the mixed solution; and redispersing the precipitate by mixing the precipitate with a saline solution.

Referring to FIG. 3, the multiple analysis device 100 according to an example of the inventive concept includes a magnetophoresis separation part MP and a magnetic field measuring part MR. The magnetophoresis separation part MP includes a channel CH where the mixed solution flows in a first direction X. A mixed solution inlet IP1 where the mixed solution is injected and a first saline solution inlet IP2 are connected to a side of the channel CH. A first outlet passageway OP1 and a second outlet passageway OP2 are connected to the other side of the channel CH.

The mixed solution includes a first kind material particle PS1 which may be a normal cell, a second kind material particle PS2 which may be a cancer cell A, and a third kind material particle PS3 which can be a cancer cell B. Although there are two kinds of the cancer cells in this example, three or more kinds thereof are possible.

Referring to FIGS. 1 and 3, the mixed solution is injected into the mixed solution inlet IP1, and a saline solution is injected into the first saline solution inlet IP2. Using a magnetophoresis method, normal cells PS1 and cancer cells PS2 and PS3 in the blood are firstly separated at the magnetophoresis separation part MP (A second step, S20). The firstly separated cancer cells PS2 and PS3 are sent to the second outlet passageway OP2 and the normal cells PS1 are exhausted through the first outlet passageway OP1.

The magnetophoresis separation part MP is explained in more details.

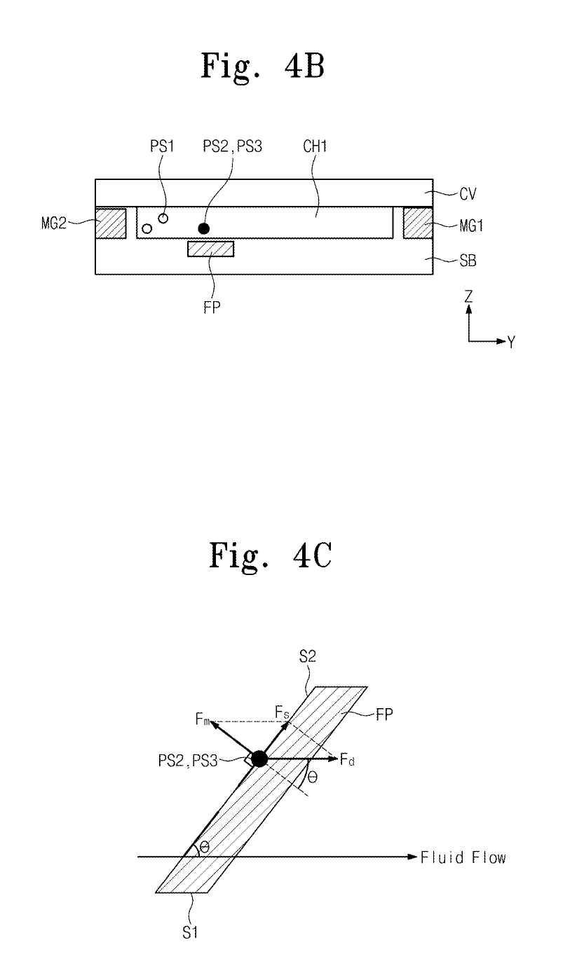

FIG. 4A is a detailed plan view of a magnetophoresis separation part contained in the multiple separation device of FIG. 2. FIG. 4B is a cross-sectional view taken along the line A-A′ of FIG. 4A. FIG. 4C shows movement of a material particle at the magnetophoresis separation part.

Referring to FIGS. 4A, 4B and 4C, at the magnetophoresis separation part MP a channel CH is disposed. At the channel CH, a mixed solution flows in a first direction X. The channel CH can be provided by a substrate SB where a groove is formed and a cover CV covering the substrate SB. Below a bottom surface of the channel CH, a ferromagnetic pattern FP is disposed. The ferromagnetic pattern FP includes a first side 51 parallel to the first direction X and a second side S2 extending closely to the second direction Y. At any position on the second side S2, a width of the ferromagnetic pattern FP parallel to the first direction X can be constant. That is, the ferromagnetic pattern FP can have a parallelogram shape in a plan view. The ferromagnetic pattern FP has a constant magnetic force at any position on the second side S2. In order to constantly magnetize the ferromagnetic pattern FP to keep the magnetic force of the first ferroelectric pattern FP1 constant, at least one permanent magnet MG1 and MG2 can be disposed to be adjacent to the first channel 1. The permanent magnets MG1 and MG2 may include a first permanent magnet MG1 and the second permanent magnet MG2. For example, one of the first permanent magnet MG1 and the second permanent magnet MG2 may be the north (N) pole, and the other of them may be the south (S) pole. The first permanent magnet MG1 and the second permanent magnet MG2 may be faced to each other and the channel CH is disposed therebetween. A third direction Z may be orthogonal to both the first direction X and the second direction Y.

The substrate SB, the cover CV may be formed of the same material. For example, the substrate SB and the cover CV may be formed of a material such as glass or plastic which has a low reactivity.

A mixed solution provided through the mixed solution inlet IP1 and mixed with the saline solution is sent to the channel CH. At this time, since the first kind material particle PS1 having a first magnetization magnitude of the lowest value includes almost no magnetic nanoparticle, the first kind material particle PS1 is not captured to the ferromagnetic pattern FP and flows along a low arrow AL showing a flow of the mixed solution. However, the second and third kind material particles PS2 and PS3 containing a lot of the magnetic nanoparticles are captured to the ferromagnetic pattern FP. A magnetic force Fm orthogonal to the second side S2 and a force Fd caused by the flow of the mixed solution are applied to the second and the third kind material particles PS2 and PS3 as shown in FIG. 4C. Consequently, the resultant force Fs of the magnetic force Fm and the force Fd is applied to the second and the third kind material particles PS2 and PS3, so that the second and the third kind material particles PS2 and PS3 move along the second side.

The magnetic force Fm may have a negative sign which is opposite to that of the force Fd caused by the flow of the mixed solution. The condition that the second and third kind material particles PS2 and PS3 are captured to the ferromagnetic pattern FP can be suggested by the following equation 1.

Fₘ+F_{d} cos θ<0 <Equation 1>

Therefore, as an angle θ between the second side S2 and the first direction X becomes increased, it increases a possibility that the second and third kind material particles PS2 and PS3 are not captured but passed.

Again referring to FIG. 4A, the second and third kind material particles PS2 and PS3 can move along an upper arrow AU. Therefore, the first material particle PS1 is transferred to the first outlet passageway OP1 and the second third kind material particles PS2 and PS3 can be transferred to the second outlet passageway OP2.

The magnetophoresis separation part MP should be long enough to separate the first kind material particle PS1 from the second and third kind material particles PS2 and PS3. At the magnetophoresis separation part MP, to separate the material particles with or without the magnetism may be possible.

Referring to FIGS. 1 and 3, a magnetic field measuring part MR is disposed at the second outlet passageway OP2. At the magnetic field measuring part MR, a magnetic sensor can be disposed to measure a magnetic field. The magnetic sensor may be a giant magnetoresistance sensor. The magnetic fields of the second and third kind material particles PS2 and PS3 can be continuously measured at the magnetic field measuring part MR (S30). The cancer cell represents difference in magnitude of the magnetic field of itself, by a difference of the number of the magnetic nanoparticles (or the number of the surface marker) combined to the cancer cell. Normal cells such as leukocyte, which are not perfectly separated at the magnetophoresis separation part MP, may have no magnetic nanoparticle or a very small amount of the magnetic nanoparticles which are non-specifically combined to the cancer cell. Therefore, the normal cells may have a very weak magnetic field. Therefore, these normal cells can be easily discriminated from the cancer cells by the magnitude of the measured magnetic field. That is, since the number of the magnetic nanoparticles combined to the second kind material particle PS2 is more than that of the magnetic nanoparticles combined to the first kind material particle PS1 but less than that of the magnetic nanoparticles combined to the third kind material particle PS3, the magnetic field of the second kind material particle PS2 is stronger than that of the first kind material particle PS1 and weaker than that of the third kind material particle PS3.

Referring to FIGS. 1 and 3, at an analysis/discrimination part 10 electrically connected to the magnetic field measuring part MR, the cancer cells can be analyzed/discriminated into cancer kinds by using the measured magnetic fields (S40). For example, if a measured magnetic field of a first cancer cell is strongest, the first cancer cell can be discriminated as the cancer cell B (PS3 of FIG. 2). If a measured magnetic field of a second cancer cell is weakest, the second cancer cell can be discriminated as the normal cell such as leukocyte (PS1 of FIG. 2).

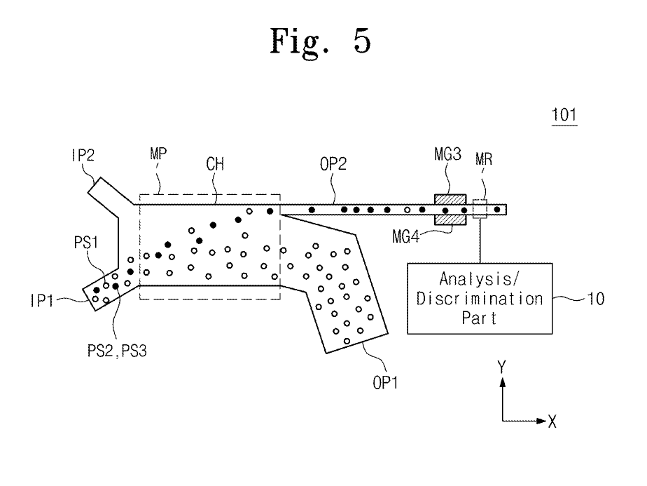

FIG. 5 is a schematic plan view showing a multiple separation device according to an example of the inventive concept.

Referring to FIG. 5, a third permanent magnet MG3 and a fourth permanent magnet MG4 can be disposed in front of the magnetic field measuring part MR to be adjacent to the second outlet passageway OP2. The third permanent magnet MG3 and the fourth permanent magnet MG4 can be faced to each other. For example, one of the third permanent magnet MG3 and the fourth permanent magnet MG4 may be the north (N) pole, and the other of them may be the south (S) pole. The third permanent magnet MG3 and the fourth permanent magnet MG4 can align directions of the magnetic fields of the magnetic nanoparticles contained in the material particles PS1, PS2 and PS3 in one direction. When the directions of the magnetic fields of the magnetic nanoparticles combined to the cancer cells are aligned in one direction, the magnitudes of the magnetic fields are strongest. Therefore, when the directions of the magnetic fields of the magnetic nanoparticles combined to the cancer cells are aligned in one direction by disposing the permanent magnets directly in front of the magnetic sensor, stronger magnetic signals can be obtained and it is possible to obtain a superior reproduction characteristic.

The positions of the permanent magnets can be variously changed, and it is possible to employ only one of the north and south poles.

In the multiple analysis device and the method of analyzing cancer cells in blood using the device according to the inventive concept, it can analyze the cancer cells along cancer kinds by using the magnetic nanoparticles combined to the markers of the cancer cells and the difference of the magnetic fields of them. It can simply pronounce a diagnosis with respect to existence or non-existence of cancer and also can discriminate cancer kinds. Furthermore, since it can almost perfectly remove interference by blood corpuscle cells, it can remarkably improve specificity than other technologies.

The above-disclosed subject matter is to be considered illustrative and not restrictive, and the appended claims are intended to cover all such modifications, enhancements, and other embodiments, which fall within the true spirit and scope of the inventive concept. Thus, to the maximum extent allowed by law, the scope of the inventive concept is to be determined by the broadest permissible interpretation of the following claims and their equivalents, and shall not be restricted or limited by the foregoing detailed description.

What is claimed is:

1. A multiple analysis device comprising:
a magnetophoresis separation part comprising:
a solution inlet where a solution is injected,
a saline solution inlet where a saline solution is injected,
a channel connected to both the solution inlet and the saline solution inlet,
at least one ferromagnetic pattern disposed below a bottom of the channel and extending in a direction crossing a flow of the solution,
a first outlet passageway where a first kind material particle contained in the solution is output, connected to the channel, and
a second outlet passageway where a second kind material particle is output, connected to the channel;
a magnetic field measuring part which measures a magnetic field of the second kind material particle and is connected to the second outlet passageway; and
an analysis/discrimination part which analyzes a magnetic field of the second kind material and discriminates the second kind material particle.

2. The device of claim 1, wherein the second kind material particle has a second magnetization magnitude and the first kind material particle has a first magnetization magnitude smaller than the second magnetization magnitude.

3. The device of claim 2, wherein the solution contains a plurality of the second kind material particles whose magnetization magnitudes are bigger than the first magnetization magnitude and different from each other, and the magnetic field measuring part continuously measures magnetic fields of the second kind material particle.

4. The device of claim 3, wherein the solution is blood, the first kind material particle is a normal cell and the second kind material particles are cancer cells whose kinds are different from each other.

5. The device of claim 4, wherein each of the cancer cells includes a different number of markers.

6. The device of claim 1, further comprising at least one first permanent magnet adjacent to the channel.

7. The device of claim 6, wherein the magnetic field measuring part includes a giant magnetoresistance sensor.

8. The device of claim 6, further comprising at least one second permanent magnet disposed in front of the magnetic field measuring part and adjacent to the second outlet passageway.

9. A method of analyzing a cancer cell in blood, comprising:
mixing a blood sample for test and a magnetic nanoparticle combined with an antibody which can specifically be reacted to a cancer cell, thereby forming a mixed solution containing a cancer cell combined with the magnetic nanoparticle;
separating the mixed solution into normal cells and cancer cells using a magnetophoresis method;
continuously measuring magnetic fields of the cancer cells; and
analyzing/discriminating the cancer cells into cancer kinds by using the magnetic fields of the cancer cells.

10. The method of claim 9, further comprising aligning directions of the magnetic fields of the magnetic nanoparticles combined to the cancer cells by using a permanent magnet, before continuously measuring the magnetic fields of the cancer cells.

11. The method of claim 9, further comprising removing a magnetic nanoparticle which is not combined to the cancer cell.

12. The method of claim 11, where the removing of a magnetic nanoparticle which is not combined to the cancer cell comprises:
obtaining a precipitate by centrifuging the mixed solution; and
redispersing the precipitate by mixing the precipitate with a saline solution.
